(19) United States
(12) Patent Application Publication
(10) Pub. No.: US 2021/0137775 A1
(43) Pub. Date: May 13, 2021

(54) MASSAGER

(71) Applicant: LG HOUSEHOLD & HEALTH CARE LTD., Seoul (KR)

(72) Inventors: Eun Mi KIM, Seoul (KR); Seon Ung MIN, Seoul (KR); Kyung Won KIM, Seoul (KR)

(73) Assignee: LG HOUSEHOLD & HEALTH CARE LTD., Seoul (KR)

(21) Appl. No.: 16/943,886

(22) Filed: Jul. 30, 2020

(30) Foreign Application Priority Data
Nov. 13, 2019 (KR) 10-2019-0145344

(51) Int. Cl. A61H 15/00; A61H 15/02

(52) U.S. Cl. CPC A61H 15/0092; A61H 2201/10; A61H 2015/0042; A61H 15/02

(57) ABSTRACT

A massager may include: a handle; a pair of roller shafts provided on left and right sides of a first side in a longitudinal direction of the handle such that a distance between the roller shafts increases towards a distal end; a roller rotatably supported on the roller shaft; a center shaft provided on the first side in the longitudinal direction of the handle to be oriented in a direction from left to right between the pair of roller shafts; and an auxiliary roller rotatably supported on the center shaft.

### CROSS-REFERENCE TO RELATED APPLICATION

The present application claims priority under 35 U.S.C. § 119(a) to Korean patent application number 10-2019-0145344, filed on Nov. 13, 2019, in the Korean Intellectual Property Office, the entire disclosure of which is incorporated herein by reference.

### BACKGROUND

### 1. Technical Field

Various embodiments of the present disclosure generally relate to a massager, and more particularly, to a massager that can be used in common without being limited to a specific user and can improve physical effects.

### 2. Related Art

Generally, a skin massager (massage device) is a beauty treatment device that massages the skin of the face using a spherical ball, and includes a handle and a pair of rollers provided on a first end of the handle. The pair of rollers is rotatably mounted on the handle as follows: a hole may be formed in a proximal end of each roller, and the roller may be supported on a roller shaft via a bearing member.

Such a massager may help improve blood circulation, because skin is stimulated by the roller as a user holds the handle by hand and then moves the roller while pressing the roller against the skin.

Furthermore, the skin massager is advantageous in that it has a size capable of holding and using the massager by one hand, so that it is easier to carry and more convenient to use, as compared with a device having the function of massaging or pressing the body such as the shoulder.

However, the skin massager for massaging the face is limited to only a pressing function by the pair of rollers as a physical function, so that efforts to improve massage effects are continuously made.

### SUMMARY

Various embodiments of the present disclosure are directed to massager capable of improving physical massage effects performed by a pair of rollers.

An embodiment of the present disclosure may provide for a massager including: a handle; a pair of roller shafts provided on left and right sides of a first side in a longitudinal direction of the handle such that a distance between the roller shafts increases towards a distal end; a roller rotatably supported on the roller shaft; a center shaft provided on the first side in the longitudinal direction of the handle to be oriented in a direction from left to right between the pair of roller shafts; and an auxiliary roller rotatably supported on the center shaft.

The auxiliary roller may be provided to press a compressed central portion, when the pair of rollers compresses skin by a movement of the handle in a front-rear direction.

The handle may include convex parts formed by outwardly protruding portions on which the roller shafts are provided, and the center shaft may connect the pair of convex parts to each other.

The auxiliary roller may be provided above an imaginary line connecting equator portions of the pair of rollers to each other in the shortest distance.

An angle between the roller shafts spread from left to right in a front-rear direction from a second side, which is opposite to the first side of the handle on which the roller is provided, to the first side may be 90 to 110 degrees, and an angle between the roller shafts spread from left to right in an up-down direction from a proximal end connected to the handle to a distal end that is a free end may be 65 to 90 degrees.

A transverse minimum spacing distance between the pair of rollers may range from 35 to 47 mm.

The center shaft may include a plurality of center shafts that are provided on central portions of the first and second sides in a longitudinal direction of the handle to be oriented from left to right, and the auxiliary roller may include a plurality of auxiliary rollers that are provided to be supported, respectively, on the center shafts provided on the first and second sides of the handle.

A distance between the auxiliary roller provided on the first side of the handle and the auxiliary roller provided on the second side of the handle may be 45 to 60 mm.

The handle may be shaped such that a vertical center line thereof may be convexly curved upwards between the first side and the second side.

The roller may have an elliptical shape, and an end of the roller in which an end of the roller shaft is located may be formed flat.

The auxiliary roller may be formed in a disc shape, with at least one protrusion being formed on a surface of the auxiliary roller coming into contact with the body.

The massager may further comprise a stimulator configured to form the roller coming into contact with a user's body and a portion of the handle gripped by the user in a circuit, thus imparting a low frequency to the roller.

### BRIEF DESCRIPTION OF THE DRAWINGS

FIG. 1 is a perspective view illustrating a bottom of a massager in accordance with a first embodiment of the present disclosure.

FIG. 2 is a perspective view illustrating a top of the massager in accordance with the first embodiment of the present disclosure.

FIG. 3 is an exploded perspective view illustrating the massager in accordance with the first embodiment of the present disclosure.

FIG. 4 is a plan view illustrating the massager in accordance with the first embodiment of the present disclosure.

FIG. 5 is a front view illustrating the massager in accordance with the first embodiment of the present disclosure.

FIG. 6 is a side view illustrating the massager in accordance with the first embodiment of the present disclosure.

FIG. 7 is a diagram illustrating another shape of an auxiliary roller of the massager in accordance with the first embodiment of the present disclosure.

FIG. 8 is a diagram illustrating the use of the massager in accordance with the first embodiment of the present disclosure.

FIGS. 9 and 10 are diagrams illustrating the use of users having different sizes, as compared with FIG. 8.

FIG. 11 is a front view illustrating a massager in accordance with a second embodiment of the present disclosure.

FIG. 12 is a front view illustrating a massager in accordance with a third embodiment of the present disclosure.

FIG. 13 is a front view illustrating a massager in accordance with a fourth embodiment of the present disclosure.

FIG. 14 is a front view illustrating a massager in accordance with a fifth embodiment of the present disclosure.

FIG. 15 is a front view illustrating a massager in accordance with a sixth embodiment of the present disclosure.

FIG. 16 is a diagram conceptually illustrating how electricity is transferred to a roller of a massager in accordance with an embodiment of the present disclosure.

FIG. 17 is a diagram conceptually illustrating a state in which components forming the electrical connection of a massager in accordance with an embodiment of the present disclosure are separated.

FIG. 18 is a diagram conceptually illustrating the components forming the electrical connection of the massager in accordance with the embodiment of the present disclosure.

### DETAILED DESCRIPTION

The above and other objects, features, and other advantages of the present disclosure will be more clearly understood from the following detailed description when taken in conjunction with the accompanying drawings. In the present specification, it should be noted that the same reference numerals are used to denote the same components throughout different drawings. In the following description, the detailed description of known functions and configurations that may unnecessarily obscure the subject matter of the present disclosure will be omitted.

Hereinafter, preferred embodiments of the present disclosure will be described in detail with reference to the accompanying drawings.

FIGS. 1 and 2 are perspective views illustrating a massager in accordance with a first embodiment of the present disclosure, FIG. 3 is an exploded perspective view illustrating the massager in accordance with the first embodiment of the present disclosure, and FIGS. 4 to 6 are diagrams illustrating any one side of the massager in accordance with the first embodiment of the present disclosure. FIG. 7 is a diagram illustrating another shape of an auxiliary roller of the massager in accordance with the first embodiment of the present disclosure.

Furthermore, FIGS. 8 to 10 are diagrams illustrating the use of the massager by users, in accordance with the first embodiment of the present disclosure.

FIGS. 11 to 15 are diagrams illustrating different embodiments, which will be described later.

FIGS. 16 to 18 are diagrams conceptually illustrating the transfer of electricity to a roller of a massager and components therefor in accordance with an embodiment of the present disclosure.

Referring to FIGS. 1 to 10 and FIGS. 16 and 18, a massager 100 in accordance with the first embodiment of the present disclosure may include a handle 110, a roller shaft 120, a roller 130, a center shaft 140, an auxiliary roller 150, and a stimulator 160. Thus, massage may be performed by a pair of rollers 130 and acupressure treatment may be further performed by the auxiliary roller 150, thus enhancing massage effects, and electrical massage effects may be achieved by the stimulator 160.

Hereinafter, in order to describe the use of the massager depending on a change in thickness of a user's calf 10 coming into contact with the pair of rollers 130, this description is based on the premise that the acupressure treatment is performed by the same strength of force. This is for helping understand the embodiment. The acupressure strength may be adjusted by a pressing force, so that the area of flesh fitted between the pair of rollers 130 may have an error range. Thus, it should be noted that this description covers all design ranges considering this error range, so that the error range of about 10% may be further included from values that will be described below.

A minimum distance of a section of an upper portion of the calf 10 that will be described below is for describing a portion of the calf 10 with which the pair of rollers 130 comes into contact. This does not mean that the entire circumference of the calf 10 is in contact with the rollers 130, so that the minimum distance may not mean the entire length of the circumference of the calf 10 but may mean the shortest distance of the calf 10 positioned between the left roller 130 and the right roller 130. Here, the shortest distance may correspond to arrows shown in FIGS. 8 to 10 that will be described below.

In addition, this is illustrated for convenience of understanding and explanation. If the flesh of the calf 10 is fitted between the pair of rollers 130 and receives acupressure treatment by the auxiliary roller 150, only a portion of the calf 10 may be fitted between the pair of rollers 130. Thus, various fitting shapes are possible.

First, the handle 110 may be held by a user, and a convex part 111 may be formed thereon to allow the roller 130 to be easily installed.

For example, the handle 110 may be configured to be easily gripped with one hand, so that the handle is longer than the width of an adult's hand. Further, in order to prevent the hand from being excessively opened, a central portion of the handle 110 may be formed to be narrow. A front portion that is the first end of the handle 110 is larger in area than a rear portion that is the second end, thus making it easy to install the roller 130.

However, such a shape of the handle 110 allows the massager to be easily held and allows the roller 130 to be easily installed. Various modifications are possible as long as they do not conflict with this embodiment. For example, the handle 110 may be formed with the same width from the first end to the second end, and may have shapes that are known to those skilled in the art.

Furthermore, the handle 110 may be formed to have a curved surface in a vertical direction so as to prevent a user's hand from interfering with skin that is massaged. That is, the handle 110 may be shaped such that its vertical center line is convexly curved upwards between a first side and a second side.

The convex part 111 of the handle 110 may be formed to protrude from the first end of the curved handle 110, so that the roller 130 may be installed thereon. Thus, since the convex part 111 protrudes from an upper surface of the handle 110 coming into contact with a user's palm to a lower surface that is opposite to the upper surface (the convex part may protrude forwards from a lower portion), the user's hand may be spaced apart from the roller 130 by the protruding distance of the convex part 111 on which the roller 130 is provided, thus preventing interference between the hand that holds the handle 110 and the body that is massaged.

The convex part 111 may be formed by outwardly protruding a portion on which the roller shaft 120 is provided. Thus, a pair of convex parts 111 may be formed to install a pair of roller shafts 120 on both sides of the first end of the handle 110, thus making it easy to install the roller shafts 120.

Therefore, the shape of the upper portion that is opposite to a portion on which each roller shaft 120 is formed is not limited to a specific shape. Thus, as described above, the massager 100 may be formed such that the center of the upper portion thereof has a convex structure and the upper portion of the first end thereof has a single surface, such as a curved surface or a flat surface. As such, various shapes are possible.

In addition, the roller shafts 120 may be installed on the convex parts 111, and the center shaft 140 may be installed between the pair of convex parts 111, so that a space may be formed between the pair of convex parts 111 to accommodate the auxiliary roller 150 installed at the center shaft 140. Thus, a portion between the convex parts 111 protruding to both sides may be a concave structure from the lower portion to the upper portion.

In other words, the handle 110 may have the convex parts 111 on both sides of a surface of the handle 110, so that the handle 110 may have various structures that allow a user to easily grip, and may be configured to install both the rollers 130 and the auxiliary roller 150. In addition, since the auxiliary roller 150 is installed at the center shaft 140 and the space is created between the pair of convex parts 111, the auxiliary roller 150 may be installed without interfering with the convex part 111. Thus, the arrangement of the pair of rollers 130 and the auxiliary roller 150 may be optimized, which will be described later but is briefly mentioned here.

Such a handle 110 is not limited to a specific material, and may be injection-molded using an ABS material, for example, which is harmless to a human body. However, the material and shape of the handle 110 may be variously changed to allow the stimulator 160 that will be described below forms a portion of the handle 110 gripped by the user to a circuit.

The handle 110 may be composed of upper and lower housings 110A and 110B that are separated in a vertical direction to allow circuit components of the stimulator 160 to be installed therein. In order to couple the upper and lower housings 110A and 1106 to each other, a boss, a rib, a coupling protrusion or the like may be formed. Since they are known to those skilled in the art, a detailed description thereof will be omitted. A flat board structure (unlabelled) on which a circuit board 162 is mounted may be provided in the handle 110 under a button 161.

In addition, the handle 110 may occupy most of an entire appearance of the massager 100 (a portion excluding the roller 130 and the auxiliary roller 150), so that a battery may be detachably provided therein or a cord may be connected thereto.

The roller shaft 120 may be provided on the convex part 111 so that the roller 130 is rotatably installed thereon. A pair of roller shafts may be provided on left and right sides of a first side in a longitudinal direction of the handle 110 such that a distance between the roller shafts increases towards a distal end. In order to transmit voltage from the stimulator 160 to the roller 130, at least a portion of the roller shaft 120 may be made of a conductive material. The roller shaft may be provided with a bearing 121 to support the rotation of the roller 130, and may electrically connect the stimulator 160 and the roller 130.

However, the roller shaft 120 may not electrically connect the stimulator 160 and the roller 130, but the stimulator 160 and the roller 130 may be directly electrically connected to each other, so that the roller shaft 120 may be made of materials other than metal. As such, various modifications are possible. The electrical connection of the roller shaft 120 will be described below.

Further, as illustrated in FIG. 4 (plan view), an angle between the roller shafts 120 that are spread from left to right in a front-rear direction from the second side, which is opposite to the first side of the handle 110 on which the roller 130 is provided, to the first side in a direction parallel to a user's skin surface may be 90 to 110 degrees, and preferably 95.25 degrees.

Furthermore, as illustrated in FIG. 5 (front view), an angle between the roller shafts that are spread from left to right in an up-down direction from the proximal end connected to the handle 110 to the distal end that is a free end in a direction perpendicular to the user's skin surface may be 65 to 90 degrees, and preferably 72.77 degrees.

Such an angle between the roller shafts 120 allows both adult men and women to use the massager regardless of the size of the calf 10, for example, without being limited to a specific body size, when flesh fitted between the pair of elliptical rollers 130 receives the acupressure treatment by the auxiliary roller 150.

That is, since the roller shafts 120 equipped with the roller 130 are spread and the pair of rollers 130 is likewise spread by the roller shafts 120, spacing distances of the rollers 130 between the proximal end and the free end of each roller shaft 120 may be different from each other. Thus, even if the body size is different, the body may come into contact with any area of each roller 130 having a spacing distance corresponding to the body size.

In addition, since the roller 130 is rotatably provided but the roller shaft 120 need not rotate, the roller shaft 120 may be configured such that threads are formed on the proximal end thereof and the roller shaft is fastened to the convex part 111 in a screw-type fastening method. Thus, the loosening of the roller shaft 120 is prevented by a washer.

As another example, a ring structure and/or a fastening groove to which a screw is coupled may be provided on a side of the roller shaft 120, and a projection to which the ring of the roller shaft 120 is connected and/or a coupling groove corresponding to the fastening groove may be provided on the handle 110, so that the roller shaft 120 and the handle 110 may be coupled to each other by the screw or the like. Alternatively, the roller shaft 120 may be coupled to the handle 110 through various coupling methods, for example, an interference fitting method or an adhesive bonding method. A known shaft coupling method of the massager may be applied.

That is, any structure that rotates the roller 130 while transferring electricity to the roller 130 may be applied without limitation. Thus, the roller shaft 120 itself may be fastened to the handle 110 by bolting or the like, or a separate protrusion (it may at least partially have a plane for bolting/screw fastening) may be provided on the roller shaft 120, so that the roller shaft 120 may be coupled to the handle 110 through various coupling methods, for example, a screw fastening method using the plane of the protrusion.

The roller 130 is configured to come into direct contact with the skin and perform massage when a user puts the massager 100 against the body. Since the roller 130 is rotatably supported on the roller shaft 120 and is rotated due to friction between the roller 130 and the skin, it prevents the skin from rubbing. Although acupressure strength for pressing the skin may be adjusted according to the strength of a user's pressing force, this embodiment will be described on the premise that the pressing strength is the same and the contact area of the roller 130 is varied depending on the size of the calf 10, as mentioned above.

The pair of rollers 130 may be provided to gather flesh therebetween. The rollers 130 may gather the flesh to perform the acupressure treatment using the auxiliary roller 150 that will be described below.

However, in order to make both the rollers 130 and the auxiliary roller 150 simultaneously come into contact with the skin, the thinner the user's calf 10 is, the shorter the spacing distance between the pair of rollers 130 should be, thus allowing the skin to come into contact with the pair of rollers 130. Further, the thicker the user's calf 10 is, the longer the spacing distance between the pair of rollers 130 should be, thus allowing the skin to come into contact with the pair of rollers 130. In other words, if the thickness of the calf 10 varies for each user, the spacing distance between the pair of rollers 130 should vary, thus allowing the rollers to come into contact with the calves 10 of various sizes.

However, if several massagers 100 are manufactured to form the spacing distance between the pair of rollers according to various body sizes, namely, several molds of different sizes are formed for individual user customization, manufacturing cost may be increased, and a cost burden for a user to purchase the massager 100 may be increased.

Thus, according to this embodiment, the pair of rollers 130 may be installed at the roller shafts 120 that are spread to both sides so that spacing distances between the first and second ends may be different from each other according to a position. Further, each roller 130 has an elliptical shape such that a contact area is increased in a longitudinal direction. Consequently, several users can use one kind of massager 100 in common.

That is, as mentioned above, the rollers 130 of this embodiment are configured such that the roller shafts 120 are spread, and thereby spacing distances from the proximal ends to the free ends of the roller shafts 120 are different from each other. Thus, entry distances of the calf to the proximal end of each roller shaft 120 are different according to the size of the calf 10, but the calf 10 may come into contact with the rollers 130. Since each roller 130 has an elliptical shape with a major axis and the major axis is aligned with the roller shaft 120, a length of the elliptical roller may be larger than that of the spherical roller, thus allowing a contact area between the roller 130 and the user's skin to be increased. Thus, this allows the calves of all users who have the thin calf 10, the mid-sized calf 10, and the thick calf 10 to come into sufficient contact with the pair of rollers 130.

For example, in order to perform the massage function regardless of the thickness of the user's calf 10 located between the pair of rollers 130, the spacing distance between the pair of rollers 130 may be set to allow all adults having the minimum distance of the section of the upper portion of the calf 10 ranging from 42 mm to 65 mm to use the massager.

To this end, the transverse minimum spacing distance between the pair of rollers 130 may range from 35 to 47 mm and preferably about 38.95 mm. Here, the entry distance of the user's calf 10 coming into contact with the pair of rollers 130 to the auxiliary roller 150 may be set according to the thickness of the calf 10.

Furthermore, according to this embodiment, the calf 10 may enter between the rollers 130 while coming into contact with the auxiliary roller 150. Thus, as portions of the rollers 130 come near to the auxiliary roller 150, a distance between the portions of the rollers 130 is reduced. In contrast, as portions of the rollers become away from the auxiliary roller 150, a distance between the portions of the rollers 130 is increased. In this case, an acupressure force may be varied by a contact distance with the auxiliary roller 150 according to the entry distance of the calf 10, but all calves may receive the acupressure treatment regardless of the thickness of the calf 10.

In addition, referring to FIG. 8 in contrast to FIGS. 9 and 10, in the case of a person having the thin calf 10 which is about 42.26 mm in the minimum distance of the section of the upper portion of the calf 10, his or her calf 10 may come into contact with the pair of rollers 130 while being located near to the proximal end of the roller shaft 120. In this case, the user's calf 10 may also come into contact with the auxiliary roller 150.

Furthermore, referring to FIGS. 9 and 10, in the case of a person having the calf which is about 50.63 mm or 64.31 mm in the minimum distance of the section of the upper portion of the calf 10, the calf may be massaged in an area of the roller 130 adjacent to the free end that is relatively distant from the proximal end of the roller shaft 120, as compared with a person having the thin calf 10. When the same pressing force is applied, the entry distance of the user's calf 10 to the auxiliary roller 150 is different from that of the thin calf 10, so that pressing degree may be varied but the calf 10 may still receive proper acupressure treatment.

Meanwhile, the acupressure strength may be adjusted according to a user's pressing force. Here, the entry distance of the calf 10 to the auxiliary roller 150 may partially adjust a flesh gathering degree according to the user's pressing force.

Such a roller 130 may physically perform acupressure and/or massage effects by the pressing of a user as well as the auxiliary roller 150. EMS electrostimulation may be performed by the stimulator 160 which will be described later, so that electrostimulation may also be performed.

To this end, the roller 130 may contain a conductive metal material, so that the entire roller may be made of metal or rubber containing metal, and current may be conducted through plating containing IPG. The roller 130 may have a flat structure which is cut so that an edge (vertex) is removed from a lower end, thus allowing the EMS current to be evenly distributed.

In addition, as mentioned above, the roller 130 may be electrically connected to the stimulator 160 via the roller shaft 120. Otherwise, the roller 130 may be directly connected to the stimulator 160. For example, a groove may be formed in an end of the roller 130 adjacent to the handle 110, and a spring (not shown) made of a metal material may be provided in the groove, so that the spring always comes into contact with the stimulator 160 and thereby an electrical connection may be made therebetween.

Moreover, the entire shape of the roller 130 may have an elliptical shape, current may not be biased to one side and may flow evenly on an entire surface of the roller 130 excluding an end, and the entire surface of the roller 130 excluding the end may be surface-treated to have uneven surfaces of various shapes such as a triangle or a rectangle, thus improving massage effects.

The center shaft 140 may be provided on the first side in the longitudinal direction of the handle 110 so that its length is formed from left to right between the pair of roller shafts 120, and may be provided to connect the pair of convex parts 111 to each other.

A plurality of center shafts 140 may be provided. For example, the center shafts may be provided, respectively, on the central portions of the first and second sides in the longitudinal direction of the handle 110 such that their lengths are formed from left to right, thus allowing the auxiliary roller provided on the center shaft 140 to rotate forwards and backwards.

The auxiliary roller 150 may be rotatably supported on the center shaft 140, so that the auxiliary roller 150 may be rotated forwards and backwards by the center shaft 140 that is transversely provided between the pair of roller shafts 120.

Such an auxiliary roller 150 may be provided to press a compressed central portion, when the pair of rollers 130 gathers or compresses the skin by the movement of the handle 110 in the front-rear direction, thus stimulating a central gastrocnemius that are central muscles of the calf 10 and venae membri inferioris, and thereby allowing blood to be smoothly circulated and relieving swelling.

The auxiliary roller 150 may be formed in a disc shape having a smaller width as compared with a spherical shape, thus making it easy to install the auxiliary roller 150 between the pair of rollers 130 and allowing acupressure force to be smoothly generated.

Furthermore, referring to FIG. 5, the auxiliary roller 150 may be provided above an imaginary line connecting equator portions of the pair of rollers 130 to each other in the shortest distance, thus allowing the user's calf 10 to come into contact with the roller 130 while being located adjacent to the proximal end of the roller shaft 120 connected to the handle 110. Thus, as mentioned above (see FIG. 8), the thin calf 10 that is about 42.26 mm in the minimum distance of the section of the upper portion of the calf 10 may also receive the acupressure treatment by the auxiliary roller 150 while flesh being gathered between the pair of rollers 130.

Meanwhile, one or more auxiliary rollers 150 may be provided, so that the auxiliary roller 150 may not only be provided on the center shaft 140 that is provided between the pair of rollers 130 on the first side of the handle 110, but also be provided to be supported by the center shaft 140 provided on the second side of the handle 110.

For example, referring to FIG. 6, a distance between the auxiliary roller 150 provided on the first side of the handle 110 and the auxiliary roller 150 provided on the second side of the handle 110 may be 45 to 60 mm, and preferably 54.50 mm.

Here, referring to FIG. 7, one or more protrusions 151 may be formed on one auxiliary roller 150 (or both of two auxiliary rollers 150). The protrusions 151 may be formed on a circumferential surface of the auxiliary roller 150 that is in contact with the body so as to enhance the acupressure effect.

For example, in order to allow a user to selectively use the protrusion 151, namely, to omit the acupressure treatment by the protrusion 151 when the pair of rollers 130 is used, the protrusion 151 may not be provided on the auxiliary roller 150 that is provided on the first side of the handle 110, and a plurality of protrusions may be formed on the auxiliary roller 150 that is provided on the second side of the handle 110, so that the auxiliary roller 150 provided on the second side of the handle 110 may apply acupressure treatment to the sole.

The protrusions 151 may effectively massage the acupoints of the foot that is called the second heart because about 7,000 nerves are concentrated. Thus, the protrusions may improve blood stream, realize body balance, and relieve stress, and in addition may achieve weight-loss effects due to the improved blood stream.

However, in order to selectively use the protrusions 151, the protrusions 151 may be omitted from the auxiliary roller 150 that is provided on the first side of the handle 110. Thus, as for the auxiliary roller 150 provided on the first side of the handle 110, a surface of the auxiliary roller 150 coming into contact with the skin may be treated to have uneven surfaces of various shapes such as a triangle or a rectangle, similarly to the roller 130.

The stimulator 160 may form the roller 130 coming into contact with the user's body and a portion of the handle 110 gripped by the user (interior of the upper and lower housings 110A and 110B) in a circuit (PCB circuit, etc.), thus imparting a low frequency to the roller 130 and consequently providing an electrical massage function.

The stimulator 160 stimulates muscles at a low frequency to smoothly induce contraction and relaxation, thereby promoting blood circulation, relaxing stiff muscles, and especially imparting Electrical Muscle Stimulation (EMS) for achieving massage effects.

Furthermore, the longer the EMS time is, the longer the stimulation may be applied. EMS voltage is related to stimulation strength. That is, the higher the EMS voltage is, the larger the stimulation strength may be applied. For example, the EMS specification of the stimulator 160 according to this embodiment is as follows: output voltage may be 5 to 60 Vpeak, a frequency may be 30 to 100 Hz, and a pulse width may be 50 to 150 μs.

In order for the stimulator 160 having such an EMS specification to transmit current to the roller 130, as mentioned above, the stimulator 160 may be directly electrically connected to the roller 130 by the spring. However, it is preferable that current be sent to the roller shaft 120.

For instance, referring to FIGS. 16 to 18 (broken lines of FIG. 16 may mean the flow of current), the stimulator 160 is provided with a wire 163 extending from the circuit board 162 on which the circuit is provided, and an electrically conductive ring part 163A is provided on an end of the wire 163 connected to the roller shaft 120, so that the wire 163 may be secured by a screw 164 that passes through a hole of the ring part 163A to be fastened to the roller shaft 120 (alternatively, soldering is possible).

Here, the ring part 163A may have various shapes such as a circular shape, an elliptical shape, or a polygonal shape. As long as the wire 163 may be secured by the screw 164, the shape of the ring part 163A may be variously modified without being limited to the ring shape. In addition, the screw 164 may not only function to secure the ring part 163A but also function to connect the wire 163 thereto, thus allowing the current to flow through the roller shaft 120 to the roller 130.

Furthermore, the structure of the current transmitted to the roller 130 by the stimulator 160 is as follows: “+” current may flow through any one of the pair of rollers 130 while “−” current may flow through the other roller 130.

To this end, “+” current of the stimulator 160 and/or a power supply (battery or the like) flows through the bearing 121 of any one of the roller shafts 120 to any one of the rollers 130, so that any one roller 130 becomes a “+” electrode. Further, “−” current of the stimulator 160 and/or a power supply (battery or the like) flows through the other roller shaft 120 of the roller shafts 120 and the bearing 121 to the other roller 130, so that the other roller 130 becomes a “−” electrode.

Thus, if any one roller 130 that is the “+” electrode and the other roller 130 that is the “−” electrode simultaneously come into contact with the skin, a closed circuit is formed around the battery, so that current may be transmitted to the skin. However, the present disclosure is not limited thereto.

Such a stimulator 160 has the button 161 in an area on an outer surface of the handle 110 where a user's hand is located or an adjacent area to selectively turn on or off the EMS operation while the user performs massage, thus causing convenience to the user. And an LED (not shown) or may be provided adjacent to the button 161 to show status of the stimulator 160.

In addition, the button 161 may control frequency strength so as to adjust the voltage, frequency, waveform, etc. For example, a button for increasing a voltage level and a button for reducing a voltage level may be provided on opposite sides of the on/off switch.

Of course, since the stimulator 160 may be driven by power supplied thereto, the circuit board 162 of the stimulator 160 may be electrically connected to the battery. An electric signal generated by the stimulator 160 may be composed of one or more signals, one or more signals may have different stimulation strength, both negative and positive polarities may be formed for one signal, or the negative polarity may be formed for any one signal and the positive polarity may be formed for the other signal. As such, various modifications are possible.

In addition, the highest point and the lowest point of the output voltage generated by the stimulator 160 may be maintained for a predetermined period of time, so that a flat portion may be formed. However, the present disclosure is not limited thereto. The waveform may be formed to descend (or ascend) in a curved shape from the highest point (or the lowest point). Thus, combinations thereof are possible. The flat portion may be formed to descend and ascend in a step shape. As such, various modifications are possible.

Such a massager 100 allows the user to selectively use only the pair of rollers 130 and the auxiliary roller 150 therebetween using the first side of the handle 110, or causes both the first and second sides of the handle 110 to come into contact with the body, thus allowing all of the pair of rollers 130 and the pair of auxiliary rollers 150 to be used. Alternatively, only the auxiliary roller 150 provided on the other side of the handle 110 comes into contact with the body, thus achieving the acupressure effect. Therefore, various massage and/or acupressure effects may be realized by one massager 100.

Particularly, this embodiment is more improved than the circular roller 130 and/or the elliptical roller 130 having a larger diameter at a lower portion according to the second to sixth embodiments that will be described below. As mentioned above, it is possible to perform massage or acupressure treatment on the calf regardless of the calf thickness and/or the user's sex, thus reducing manufacturing and purchasing costs. The weighting of the center of gravity may be improved by the elliptical shape having the larger diameter at the lower portion (hereinafter, the fifth embodiment).

FIGS. 11 to 15 are diagrams illustrating massagers in accordance with the second to sixth embodiments of the present disclosure.

Here, equally or similarly to the first embodiment, the massagers 100 in accordance with the second to sixth embodiments may include a handle 110, a roller shaft 120, a roller 130, a center shaft 140, an auxiliary roller 150, and a stimulator 160. Thus, massage may be performed by a pair of rollers 130 and acupressure treatment may be further performed by the auxiliary roller 150, thus enhancing massage effects, and electrical massage effects may be achieved by the stimulator 160, thus enhancing physical effects.

The second to sixth embodiments are different from the first embodiment in terms of the shape, distance, and the like of the roller 130 and/or the auxiliary roller 150. Hereinafter, these differences will be mainly described, and a duplicated description of the same function and operation as the first embodiment will be omitted.

First, referring to FIG. 11, in the massager 100 in accordance with the second embodiment, each of the pair of rollers 130 may have a circular shape, and a minimum distance between the rollers 130 may be about 48 mm. Here, the lowermost portion of the auxiliary roller 150 may be located below the equator of the roller 130, thus making it easy to come into contact with the calf 10.

Here, unlike the above-mentioned auxiliary roller 150, two ellipses are arranged side by side in a transverse direction to make an auxiliary roller 150 of an integrated structure, so that two acupressure points may be formed in one auxiliary roller 150.

Such a massager 100 may perform acupressure treatment by the auxiliary roller 150 having two acupressure points while the flesh of the calf 10 is gathered between the pair of rollers 130.

Furthermore, in the second embodiment, the auxiliary roller 150 may be covered by a coupler of the auxiliary roller 150. In the case of a user having the calf 10 that is small or large in section of an upper portion of the calf 10, it may be difficult to come into contact with a proper position of the calf 10. Thus, in an embodiment, one elliptical auxiliary roller 150 exposed to an outside may be preferable as in the first embodiment.

The third to sixth embodiments that will be described below may have numerical values that will be described below to be optimized for a specific user. However, these embodiments may be preferably made as in the first embodiment, and these embodiments may combine with each other to constitute another modification.

Referring to FIGS. 12 and 13, the massager 100 in accordance with the third embodiment or the fourth embodiment may be configured such that a pair of rollers 130 is formed in a circular shape equally/similarly to the second embodiment, and a minimum distance between the pair of rollers 130 may be about 34 mm or about 32 mm. Here, the lowermost portion of the auxiliary roller 150 may be located below the equator of the roller 130, thus making it easy to come into contact with the calf 10.

If the minimum distance between the pair of rollers 130 is 34 mm or more, massage effects for a thin leg may be deteriorated. Meanwhile, if the minimum distance is 32 mm or less, it may be difficult to accommodate a thick leg such as a male leg.

Therefore, unlike the second embodiment, the minimum distance between the pair of rollers 130 may be made differently and the auxiliary roller 150 may be composed of one ellipse. Thus, this embodiment may be optimized for a person who is 40 mm or less in minimum distance of the section of the upper portion of the calf 10.

Referring to FIGS. 14 and 15, the massager 100 in accordance with the fifth embodiment or the sixth embodiment may be configured such that a pair of rollers 130 is formed in an elliptical shape similarly to the first embodiment, and a minimum distance between the pair of rollers 130 may be about 47 mm or about 40 mm. Here, the lowermost portion of the auxiliary roller 150 may be located above the equator of the roller 130 similarly to the first embodiment, thus making it easy to come into contact with the calf regardless of the thickness of the calf 10.

However, unlike the first embodiment, the roller 130 may be increased in size or formed in a blunt elliptical shape, so that a difference between a sectional area of a region near to each of opposite ends of the roller 130 and a diameter of the equator may be smaller than that of the first embodiment, and the surface treatment may be formed in a longitudinal direction.

Such an embodiment is suitable for performing massage and/or acupressure treatment on a body such as the calf 10 that is fatter than the face, and the acupressure treatment may be performed by the auxiliary roller 150, thus recovering from muscle fatigue, and realizing physical massage effects and electrical stimulation, thereby being effective to dissipate fatigue.

A massager in accordance with the present disclosure is advantageous in that a massage function is performed by a pair of rollers, a physical function such as acupressure effects is performed by an auxiliary roller, muscles are stimulated with a low frequency to smoothly induce contraction and relaxation, thus promoting blood circulation and relaxing stiff muscles. Since each roller has an elliptical shape and the pair of rollers is arranged to be spread laterally, the massager may come into contact with the calf regardless of the thickness of the calf as the body enters a gap between the rollers. Therefore, the massager may be used by all men and women without being limited to a specific user having the thin or thick calf, and may be applied to variously curved body regions, such as forearms, which may be in contact with a pair of rollers. Consequently, the massager may be universally used.

The present disclosure is not limited to the above-described embodiments, and combination of the above embodiments or combination of at least one of the above embodiments and known technologies may be naturally included as another embodiment.

Although the present disclosure was described with reference to specific embodiments, it is apparent to those skilled in the art that the present disclosure may be changed and modified in various ways without departing from the scope of the present disclosure, which is described in the following claims.

All simple modifications or changes of the present disclosure fall within the purview of the present disclosure, and the specific scope of the present disclosure will be apparent from the following claims.

What is claimed is:

1. A massager, comprising:
a handle;
a pair of roller shafts provided on left and right sides of a first side in a longitudinal direction of the handle such that a distance between the roller shafts increases towards a distal end;
a roller rotatably supported on the roller shaft;
a center shaft provided on the first side in the longitudinal direction of the handle to be oriented in a direction from left to right between the pair of roller shafts; and
an auxiliary roller rotatably supported on the center shaft.

2. The massager of claim 1, wherein the auxiliary roller is provided to press a compressed central portion, when the pair of rollers compresses skin by a movement of the handle in a front-rear direction.

3. The massager of claim 1, wherein the handle comprises convex parts formed by outwardly protruding portions on which the roller shafts are provided, and
the center shaft connects the pair of convex parts to each other.

4. The massager of claim 1, wherein the auxiliary roller is provided above an imaginary line connecting equator portions of the pair of rollers to each other in the shortest distance.

5. The massager of claim 1,
wherein an angle between the roller shafts spread from left to right in a front-rear direction from a second side, which is opposite to the first side of the handle on which the roller is provided, to the first side is 90 to 110 degrees, and
wherein an angle between the roller shafts spread from left to right in an up-down direction from a proximal end connected to the handle to a distal end that is a free end is 65 to 90 degrees.

6. The massager of claim 1, wherein a transverse minimum spacing distance between the pair of rollers ranges from 35 to 47 mm.

7. The massager of claim 1,
wherein the center shaft comprises a plurality of center shafts that are provided on central portions of the first and second sides in a longitudinal direction of the handle to be oriented from left to right, and
wherein the auxiliary roller comprises a plurality of auxiliary rollers that are provided to be supported, respectively, on the center shafts provided on the first and second sides of the handle.

8. The massager of claim 1, wherein a distance between the auxiliary roller provided on the first side of the handle and the auxiliary roller provided on the second side of the handle is 45 to 60 mm.

9. The massager of claim 1, wherein the handle is shaped such that a vertical center line thereof is convexly curved upwards between the first side and the second side.

10. The massager of claim 1, wherein the roller has an elliptical shape, and an end of the roller in which an end of the roller shaft is located is formed flat.

11. The massager of claim 1, wherein the auxiliary roller is formed in a disc shape, with at least one protrusion being formed on a surface of the auxiliary roller coming into contact with the body.

12. The massager of claim 1, further comprises a stimulator configured to form the roller coming into contact with a user's body and a portion of the handle gripped by the user in a circuit, thus imparting a low frequency to the roller.
